(19) United States
(12) Patent Application Publication
(10) Pub. No.: US 2010/0144046 A1
(43) Pub. Date: Jun. 10, 2010

(54) CYCLOMETALATED TRANSITION METAL COMPLEXES FOR MULTIPLEX ANALYTE DETECTION

(75) Inventor: Thomas R. Berkelman, Oakland, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA (US)

(21) Appl. No.: 12/627,393

(22) Filed: Nov. 30, 2009

Related U.S. Application Data
(60) Provisional application No. 61/120,032, filed on Dec. 4, 2008.

(51) Int. Cl. C07F 15/00; G01N 33/68

(52) U.S. Cl. 436/86; 546/4

(57) ABSTRACT

A complex containing a transition metal ion and a plurality of donor ligands each of which is fully coordinated to the transition metal ion and is either a nitrogen donor ligand or a cyclometalated donor ligand, such that at least one of the donor ligands is a cyclometalated donor ligand bears one or more reactive groups connected to at least one of the donor ligands through a linker that includes a chain of four or more atoms. The linker offers advantages that make the complex particularly effective in labeling biomolecules and in multiplex analyses.

### CROSS REFERENCE TO RELATED APPLICATION

This application claims the benefit of U.S. Provisional Patent Application No. 61/120,032, filed Dec. 4, 2008, the contents of which are incorporated herein by reference.

### BACKGROUND OF THE INVENTION

1. Field of the Invention

This invention resides in the field of luminescent labels for biomolecules, including multiplex detection systems for multiple species in a sample of biological material.

2. Background of the Invention

Sets of two or more luminescent labels that are similar to one another in their physical properties but distinct in their luminescence characteristics are known for use in multiplex formats for the detection and quantification of biomolecules. Examples of such compounds are the CyDye fluorescent labeling reagents distributed by GE Healthcare Life Sciences (Piscataway, N.J., USA). These reagents are primarily used in multiplexed microarray and immunodetection applications. A description of sets of fluorescent covalent labels that are physically matched yet optically distinguishable is found in Minden et al., U.S. Pat. No. 6,426,190 B1, issued Jul. 30, 2002. The labels in this patent are reactive cyanine-based fluorescent dyes that are identical in charge and molecular weight but distinct in their spectroscopic properties. Proteins labeled with different dyes are mixed and separated electrophoretically. The dyes are identical to one another in their effect on the electrophoretic behavior of the labeled proteins, but the various proteins can be distinguished from each other by

Luminescent labeling reagents, including the cyanine-based labeling reagents described above, are typically organic dyes with Stokes shifts (the difference in wavelength between the excitation maximum and the emission maximum) under 50 nm. Fluorescent labels that emit light in the easily imaged visible light region of the spectrum can thus be excited by light that is also in the visible region. For optimal fluorescence detection, monochromatic or narrow-band visible light is needed, and this requires lasers or other specialized light sources. In multiplexed fluorescence applications, the labels have distinct individual excitation maxima, and this requires multiple light sources which are generally available only in expensive and specialized instrumentation.

The fluorescence imaging of gels or blots is often performed with instrumentation employing illumination in the UV region of the spectrum, since UV light sources are inexpensive and readily available, and since the UV light used for excitation is easily filtered from the visible light fluorescence signal. Commonly used fluorescent labels are not optimally imaged with UV excitation, however.

Certain transition metals are known to provide luminescent complexes with the high extinction coefficient, high quantum yield and high chemical stability that are needed for labeling biomolecules. These metal complexes also have excitation maxima in the UV range and broad Stokes shifts, and are therefore uniquely suitable for use with UV-excitation based fluorescence imaging. Ruthenium(II)-based luminescent metal complexes that can be used to label polypeptides through covalent linkage are described, for example, in Terpetschnig, E., et al. (1995), “Metal-Ligand Complexes as a New Class of Long-Lived Fluorophores for Protein Hydrodynamics,” Biophysical Journal 68: 342-350; Tokarski, C., et al. (2006), “High-sensitivity staining of proteins for one- and two-dimensional gel electrophoresis using post migration covalent staining with a ruthenium fluorophore,” Electrophoresis 27: 1407-1416; Müller et al. U.S. Pat. No. 5,075,447, Dec. 24, 1991; and Bard, A. J., et al., U.S. Pat. No. 5,453,356, Sep. 26, 1995.

Iridium(III)-based cyclometalated complexes that can be used to label biological molecules through covalent linkage are described in Lo, K. K-W., et al. (2005), “Biological labelling reagents and probes derived from luminescent transition metal polypyridine complexes” Coordination Chemistry Reviews 249: 1434-1450; Lo, K. K-W., et al. (2001). “First Examples of Luminescent Cyclometalated Iridium(III) Complexes as Labeling Reagents for Biological Substrates,” Organometallics 20: 4999-5001; and Lo, K. K-W., et al. (2003), “New Luminescent Cyclometalated Iridium(III) Diimine Complexes as Biological Labeling Reagents,” Inorganic Chemistry 42: 6886-6897.

Photoluminescent cyclometalated iridium complexes have certain features that are of relevance to the design of biological labeling reagents. They have particularly high quantum yields that can be in excess of 50%. They have strong UV absorbance with maxima in the range 250-300 nm. The spectrum of their emitted light is highly dependent on the nature of the donor ligand substituents, and complexes have been described with emission maxima covering the visible spectrum from ˜450 nm to ˜650 nm. See, e.g., Lowry, M. S., et al. (2006), “Synthetically Tailored Excited States: Phosphorescent, Cyclometalated Iridium(III) Complexes and Their Applications” Chem. Eur. J. 12: 7970-7977; and De Angelis, F., et al. (2007), “Controlling Phosphorescence Color and Quantum Yields in Cationic Iridium Complexes: A Combined Experimental and Theoretical Study,” Inorganic Chemistry 46: 5989-6001.

Of further potential relevance to the present invention is International Application No. WO 2009/067603 A1, published on May 28, 2009 under the Patent Cooperation Treaty, and its United States counterpart, Published Patent Application No. US 2009/0131640 A1, published May 21, 2009, on application Ser. No. 12/274,979, filed Nov. 20, 2008.

### SUMMARY OF THE INVENTION

This invention resides in a complex containing a transition metal ion and a plurality of donor ligands each of which is fully coordinated to the transition metal ion and is either a nitrogen donor ligand or a cyclometalated donor ligand, such that at least one of the donor ligands is a cyclometalated donor ligand and at least one of the donor ligands bears one or more reactive groups connected to the donor ligand through a linker. The reactive group is one that is reactive with a functional group on a target molecule to form covalent bond, and the linker is one that includes a chain of four or more atoms. When two or more nitrogen donor ligands are present in the same complex, the nitrogen donor ligands can be the same or different. Likewise, When two or more cyclometalated donor ligands are present in the same complex, the cyclometalated donor ligands can be the same or different. The linker improves the ease of When two or more cyclometalated donor ligands are present in the same complex, the cyclometalated donor ligands can be the same or different. The linker improves the ease of bonding the reactive group to the target molecule (through the functional group on the target molecule) by reducing interference from the donor ligands in the bonding reaction, and thus produces higher yields in the attachment of the complex to the target molecule. The physical separation provided by the linker also reduces the degree of interaction between the target molecule and the complex that might affect the emission spectra of the complex upon excitation.

The invention further resides in the use of two or more such complexes in a multiplex detection system for simultaneously detecting multiple analytes, typically proteins or other biomolecules, by affinity interactions. Thus, for example, two or more samples containing proteins or nucleic acids can be directly reacted with different complexes distinguishable on the basis of the emission spectra of the donor groups on the complexes. The samples can then be mixed together and separated electrophoretically or by other forms of chromatography, and differences among the samples are detected and quantified by analyzing the luminescence emissions. Sample components unique to one sample or the other, or present in a greater ratio in one sample or the other, can be detected as a difference in luminescence emission. The “target molecules” in these cases are affinity binding members that selectively bind to individual proteins or nucleic acids in the samples. A feature of the complexes that makes them particularly useful in multiplex detection systems is that minor structural variations on one or more of the donor ligands will produce a change in the emission spectrum with little or no change in the physicochemical properties of the complex. The resulting differences in emission spectra between different complexes will be readily differentiable, thereby making multiple analytes in a common mixture clearly detectable while each remains identifiable. Using known multiplex protocols, the complexes are selected such that different complexes produce luminescent emissions that are differentiable from each other although excitable by a common irradiation band. A single sample containing multiple analytes can also be incubated with a two or more complexes, equal to or greater than the number of analytes, the complexes having immunological binding members covalently bound thereto, to bind the complexes to the analytes through affinity-type interactions. When the sample is then irradiated with excitation energy, the various spectra from the different complexes can be simultaneously detected and differentiated to provide information on the presence and amount of each analyte.

In still further embodiments, the “target molecules” to which the complexes are covalently bound are the analytes themselves. A sample with multiple analytes, or multiple samples with individual or multiple analytes are thus reacted with the complexes to form a covalent bond between each analyte and the reactive group on one of the complexes. If two or more samples are labeled in this manner, they can then be mixed together and separated electrophoretically or by other forms of chromatography, and differences among the samples are detected and quantified by analyzing the luminescence emissions. Detection of and distinguishing between the analytes are then achieved by luminescence emission as described above.

The invention further resides in a method for detecting an analyte in a sample by contacting the sample with a complex that includes (i) a transition metal ion, (ii) a plurality of donor ligands, each donor ligand fully coordinated to said transition metal ion and each donor ligand being either a nitrogen donor ligand or a cyclometalated donor ligand, such that at least one of the donor ligands is a cyclometalated donor ligand, and (iii) either an antibody, an oligonucleotide, or a small-molecule affinity-type binding member, that is interactive with the analyte upon contact to bind thereto by either a covalent bond or an affinity-type interaction and that is covalently attached to one of the donor ligands through a linker that includes a chain of 4 to 10 atoms. Once the sample is contacted with the complex, the sample is irradiated with excitation energy, and luminescence emission from the components of the sample other than unbound complex is detected as an indication of the presence of the analyte in the sample.

The present invention further resides in a kit for labeling proteins, nucleic acids, or other biological analytes, the kit containing two or more metal complexes as described above and optionally other materials. The metal complexes will be selected to be distinguishable from each other by their emission spectra, and are present in the kit either as pure substances or as stock solutions. The metal complexes in the kit are either ready for covalent coupling to affinity-type binding reagents, or are already coupled to such reagents and ready for affinity binding to analytes. The optional materials included in the kit are one or more species selected from buffers, chelators, detergents, or chemical components in general that promote the covalent bonding of the complexes to the affinity binding reagents, or the affinity-type labeling of analytes with the affinity binding reagents that are already part of the complex, or the purification of the labeled analytes. Certain kits of the present invention also include tubes, columns, or other receptacles for retaining the complex, the affinity binding reagents, the analytes, and any other chemical components of the kit to permit the labeling reaction to take place. Certain kits also contain chromatographic separation media. Certain kits contain two or more of such metal complexes having different emission spectra.

The complexes of this invention have high extinction coefficients and quantum yields and consequently allow sensitive detection, and they can be excited with inexpensive and simple UV light sources. The complexes have high photostability and thermostability, and produce emission spectra that are insensitive to pH. The complexes of this invention also have long luminescence lifetimes and can be used in applications utilizing time-resolved instrumentation. The novel linkers offer the further advantage of minimizing or eliminating steric interference in the labeling reaction. Still further, since each complex bears a single positive charge and reacts with proteins to convert a positively charged amine to a neutral amide, labeling of proteins with the complexes has minimal effect on the isoelectric points of the proteins.

Further objects, aspects, features, and advantages of the invention will be apparent from the description that follows.

### BRIEF DESCRIPTION OF THE DRAWINGS

FIG. 1 depicts fluorescence emission spectra of two structurally related complexes of the present invention.

FIG. 2 depicts the results of an experiment in which electrophoretically separated proteins labeled with the two metal complexes of FIG. 1 are distinguished from each other through the use of optical filters.

### DETAILED DESCRIPTION OF THE INVENTION AND PREFERRED EMBODIMENTS

### Linker

The linker includes a chain of atoms linked together single, double, triple or aromatic carbon-carbon bonds, carbon-nitrogen bonds, nitrogen-nitrogen bonds, carbon-oxygen bonds, carbon-sulfur bonds, phosphorus-oxygen bonds, phosphorus-nitrogen bonds, or nitrogen-platinum bonds, or combinations of these types of bonds. The chain of atoms can include ether, thioether, carboxamide, sulfonamide, urea, urethane or hydrazine moieties, or combinations thereof. In one embodiment, the covalent linkage incorporates a platinum atom, such as described in Houthoff, H. J., et al., U.S. Pat. No. 5,714,327 (Feb. 3, 1998). Preferred linkers are those including a chain of four or more atoms selected from the group consisting of C, N, O, P, and S; and are composed of any combination of ether, thioether, amine, ester, carboxamide, sulfonamide, hydrazide bonds and aromatic or heteroaromatic bonds. Certain preferred linkers are those containing both single carbon-carbon bonds and carboxamide or thioether bonds. Further preferred are those containing 4 to 10 atoms, optionally including one or two heteroatoms. Examples of linkers are polymethylene, arylene, alkylarylene, arylenealkyl, or arylthio, all either substituted or unsubstituted. Preferred classes of polymethylene linkers are C₂-C₁₀ polymethylene, C₄-C₁₀ polymethylene, and C₂-C₆ polymethylene. Other examples of linkers are those of the formulas —(CH₂)_{d}(CONH(CH₂)ₑ)_{z}—, —(CH₂)_{d}(CON(CH₂)₄NH(CH₂)ₑ)_{z}—, —(CH₂)_{d}(CONH(CH₂)ₑNH)_{z}—, and —(CH₂)_{d}(CONH(CH₂)ₑNHCO)_{z}—, where d is 0-5, e is 1-5 and z is 0 or 1.

### Reactive Group

The choice of the reactive group that joins the complex to an amino acid or a portion of any molecule to which the complex will be covalently joined in accordance with this invention will depend on the functional group on the molecule. The types of functional groups typically present on amino acids and other molecules include, but are not limited to, amines, amides, thiols, alcohols, phenols, aldehydes, ketones, phosphates, imidazoles, hydrazines, hydroxylamines, disubstituted amines, halides, epoxides, carboxylate esters, sulfonate esters, purines, pyrimidines, carboxylic acids, olefinic bonds, or combinations of these groups. A single type of reactive site may be available on the substance (typical for polysaccharides), or a variety of sites such as amines, thiols, alcohols, and phenols, for example, may occur, as is typical for proteins. A conjugated substance may be conjugated to more than one complex, which may be the same or different, or to a substance that is additionally modified by a hapten, such as biotin. Although some selectivity can be obtained by careful control of the reaction conditions, selectivity of labeling is best obtained by selection of an appropriate reactive complex. In preferred embodiments, the reactive group will react with an amine, a thiol, an alcohol, an aldehyde or a ketone. Particularly preferred reactive groups are those that react with an amine or a thiol functional group. Examples of reactive groups are acrylamides, reactive amines (such as a cadaverine or ethylenediamine), activated esters of carboxylic acids (such as succinimidyl esters of carboxylic acids), acyl azides, acyl nitriles, aldehydes, alkyl halides, anhydrides, anilines, aryl halides, aziridines, boronates, carboxylic acids, diazoalkanes, haloacetamides, halotriazines, hydrazines (including hydrazides), imido esters, isocyanates, isothiocyanates, maleimides, phosphoramidites, reactive platinum complexes, sulfonyl halides, and thiols. By “reactive platinum complex” is meant chemically reactive platinum complexes such as described in van den Berg, F. M., et al., U.S. Pat. No. 5,580,990 (Dec. 3, 1996) and Houthoff, H. J., et al., U.S. Pat. No. 5,985,566 (Nov. 16, 1999). Where the reactive group is a photoactivatable group, such as an azide, diazirinyl, azidoaryl, or psoralen derivative, the complex becomes chemically reactive only after illumination with light of an appropriate wavelength. Where the reactive group is an activated ester of a carboxylic acid, the reactive complex is particularly useful for preparing complex-conjugates of proteins, nucleotides, oligonucleotides, or haptens. Where the reactive group is a maleimide or haloacetamide, the reactive complex is particularly useful for conjugation to thiol-containing substances. Where the reactive group is a hydrazide, the reactive complex is particularly useful for conjugation to periodate-oxidized carbohydrates and glycoproteins, and in addition is an aldehyde-fixable polar tracer for cell microinjection. Preferred reactive groups are carboxylates, succinimidyl groups, such as succinimidoococarbonyl, haloacetamides, hydrazines, isothiocyanates, maleimide groups, aliphatic amines, perfluorobenzamido groups, azidoperfluorobenzamido groups, reactive platinum complexes, and psoralens. More preferred reactive groups are succinimidooxycarbonyl, maleimide, iodoacetamide, and reactive platinum complexes. Preferred reactive platinum complexes are haloplatinates or platinum nitrate. In a particularly embodiment the reactive group is a succinimidyl group, such as succinimidooxocarbonyl.

Preferred reactive groups are electrophilic groups and the preferred groups within which the reactive groups will form covalent bonds are nucleophilic groups. Examples of electrophilic groups, nucleophilic groups, and the resulting covalent linkages, are shown in Table I.

TABLE I

| Electrophilic | Nucleophilic | Covalent |
|---|---|---|
| Group | Group | Linkage |
| Activated ester (such as an | Amine | Carboxamide |
| N-hydroxysuccinimide ester | | |
| or tetrafluorophenyl ester) | | |
| Acrylamide | Thiol | Thioether |
| Acyl azide | Amine | Carboxamide |
| Acyl halide | Amine | Carboxamide |
| Acyl halide | Alcohol | Ester |
| Acyl nitrile | Amine | Carboxamide |
| Acyl nitrile | Alcohol | Ester |
| Aldehyde | Amine | Imine |
| Aldehyde or ketone | Hydrazine | Hydrazone |
| Aldehyde or ketone | Hydroxylamine | Oxime |
| Alkyl halide | Amine | Alkyl amine |
| Alkyl halide | Carboxylic acid | Ester |
| Alkyl halide | Alcohol | Ether |
| Alkyl halide | Thiol | Thioether |
| Alkyl sulfonate | Thiol | Thioether |
| Alkyl sulfonate | Carboxylic acid | Ester |
| Alkyl sulfonate | Alcohol | Ether |
| Alkyl thiosulfonate | Thiol | Disulfide |
| Anhydride | Alcohol | Ester |
| Anhydride | Amine | Carboxamide |
| Aryl halide | Thiol | Phenyl thioether |
| Aryl halide | Amine | Aryl amine |
| Azide | Alkyne | 1,2,3-triazole |
| Azide | Cycloalkyne | 1,2,3-triazole |
| Azide | Ester-substituted | Amide |
| | triaryl phosphine | |
| Aziridine | Thiol | Thioether |
| Boronate | Glycol | Boronate ester |
| Carbodiimide | Carboxylic acid | N-acylurea or anhydride |
| Diazoalkane | Carboxylic acid | Ester |
| Diene | Benzoquinone | Cyclohexene |
| Epoxide | Thiol | Thioether |
| Epoxide | Amine | Alkyl amine |
| Haloacetamide | Thiol | Thioether |
| Haloplatinate | Amine | Platinum complex |
| Haloplatinate | Heterocycle | Platinum complex |
| Haloplatinate | Thiol | Platinum complex |
| Halotriazine | Amine | Aminotriazine |
| Halotriazine | Alcohol | Triazinyl ether |
| Imido ester | Amine | Amidine |
| Isocyanate | Amine | Urea |
| Isocyanate | Alcohol | Urethane |
| Isothiocyanate | Amine | Thiourea |
| Maleimide | Thiol | Thioether |
| Phosphoramidite | Alcohol | Phosphite ester |
| Pyridyl disulfide | Thiol | Disulfide |
| Pyrilium | Amine | Alkyl pyridinium |
| Silyl halide | Alcohol | Silyl ether |
| Sulfamidate | Thiol | Thioether |
| Sulfonate ester | Amine | Alkyl amine |
| Sulfonate ester | Thiol | Thioether |
| Sulfonate ester | Carboxylic acid | Ester |
| Sulfonate ester | Alcohol | Ether |
| Sulfonyl halide | Amine | Sulfonamide |
| Sulfonyl halide | Alcohol | Sulfonate ester |
| α,β-Unsaturated carbonyl | Thiol | Thioether |

The reactive group can be joined to any donor ligand on the complex through the linker. Preferably, the reactive group is joined to the complex as a substituent on a nitrogen donor ligand.

The following formula represents a particularly preferred combination of reactive group, linker, and substituent on a donor ligand to which the linker is bonded.

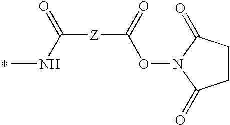

in which the asterisk denotes the point of attachment to the donor ligand, and Z is the linker. Z is either a straight-chain alkyl or polyoxyethylene group having from 2 to 20 carbon atoms and at least 2 contiguous carbon atoms. These carbon atoms are optionally substituted by groups such as hydroxy, amido, amino, alkyl sulfonic acid, alkyl phosphonic acid, and phosphoric acid. Alternatively, the succinimido can be replaced by other reactive groups such as another activated ester N-hydroxysulfosuccinimidyl ester, 1-oxybenzotriazolyl ester, tetrafluorophenyl ester or any ester formed with an aryloxy group or aryloxy substituted one or more times by electron withdrawing substituents such as nitro, fluoro, chloro, cyano, or trifluoromethyl, or combinations thereof, acrylamide, acyl azide, acyl halide, acyl nitrile, aldehyde, alkyl halide, alkyl sulfonate, anhydride, aryl halide, azide, aziridine, benzophenone, boronate, carbodiimide, diazoalkane, epoxide, ester-substituted triaryl phosphine, haloacetamide, haloplatinate, halotriazine, imido ester, isocyanate, isothiocyanate, ketone, maleimide, phosphoramidite, pyridyl disulfide, pyrilium, silyl halide, sulfonate ester, sulfonyl halide, vinyl sulfone, vinyl pyridine, amine, aniline, alkyne, cycloalkyne, alcohol, phenol, hydrazine, hydroxylamine, carboxylic acid, thiol, selenol, glycol, or any other group capable reacting to form a covalent linkage with a target.

### Donor Ligands

As used herein, the term “donor ligand” refers to a ligand that donates one or more of its electrons through a coordinate bond (where both electrons shared in a bond come from the same atom) to one or more central atoms or ions, which in the present invention is a transition metal ion. The electrons from the donor ligand can be lone pairs of electrons, such as on a nitrogen, oxygen, sulfur or phosphorus, for example. Alternatively, the electrons from the donor ligand can be from an anion, such as a carbanion and an oxygen anion. When the lone pair of electrons is donated from only nitrogen atoms, then the ligand is a nitrogen donor ligand. Multidentate ligands are ligands containing more than one atom coordinately bonded to a single transition metal ion. When the donor ligand is multidentate and at least one coordinating atom is carbon such that a covalent bond is formed between the metal and the carbon, then the ligand is a cyclometalated ligand. Cyclometalated complexes within the scope of this invention are complexes between at least one cyclometalated ligand and at least one transition metal ion. Cyclometalated complexes are formed through the loss of a proton at the site of carbon-metal coordination and a change in the charge of the complex of −1 per carbon-metal bond formed. Cyclometalated ligands are therefore formally described as comprising a carbanion at the site of carbon-metal coordination. One of skill in the art will appreciate that other donor ligands are also useful in the present invention.

### Nitrogen Donor Ligands

Nitrogen donor ligands useful in the method of the present invention include any nitrogen donor ligand that has two nitrogen donor atoms available to bind to a metal. The metal complexes of the present invention can have any number of nitrogen donor ligands. In some embodiments, the metal complexes can have 0, 1 or 2 nitrogen donor ligands. When two or more nitrogen donor ligands are present in a single metal complex, they can be the same or different. Preferred nitrogen donor ligands are those containing a heteroaryl ring system having from ten to forty ring atoms, wherein from two to eight of the ring atoms are heteroatoms, the heteroatoms being N, O, S, or combinations thereof, and at least two of the heteroatoms are N. The nitrogen donor ligand is substituted with from zero to four R¹ groups, where R¹ is either C₁-C₆ alkyl, C₂-C₆ alkenyl, C₂-C₆ alkynyl, C₁-C₆ alkoxy, halogen, C₁-C₆ haloalkyl, —OR², —NR²R³, —CN, —C(O)R², —C(O)OR², —OC(O)R², —C(O)NR²R³, —N(R²)C(O)R³, —OC(O)NR²R³, —N(R²)C(O)OR³, —NR²C(O)NR³R⁴, —NR²C(O)NR³R⁴, —NR²C(S)NR³R⁴, —NO₂, ═O, aryl, heteroaryl, cycloalkyl, heterocycloalkyl, carboxylate, alkyl carboxylate, aryl carboxylate, sulfonic acid, alkyl sulfonic acid, aryl sulfonic acid, phosphonic acid, alkyl phosphonic acid, or aryl phosphonic acid. In the above groups, R² is H or C₁-C₁₂ alkyl, R³ is H or C₁-C₁₂ alkyl, and R⁴ is H or C₁-C₁₂ alkyl.

Preferred heteroaryl ring systems for the nitrogen donor ligand are those having ten to thirty ring atoms and in which each heteroatom is N. Further preferred heteroaryl ring systems are fused ring systems having ten to twenty ring atoms and in which each heteroatom is N and R¹ is halogen.

Examples of generic structures for nitrogen donor ligands are as follows:

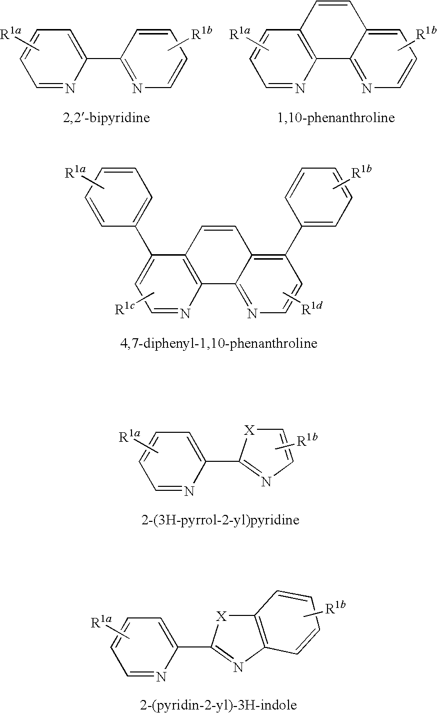

in which each of R^{1a}, R^{1b}, R^{1c} and R^{1d} are as defined above for R¹, and X is an optionally substituted methylene, O, S, —NR² or Se.

Examples of specific nitrogen donor ligands are as follows, on some of which are shown the ligand and reactive group, represented by “L” and “G,” respectively:

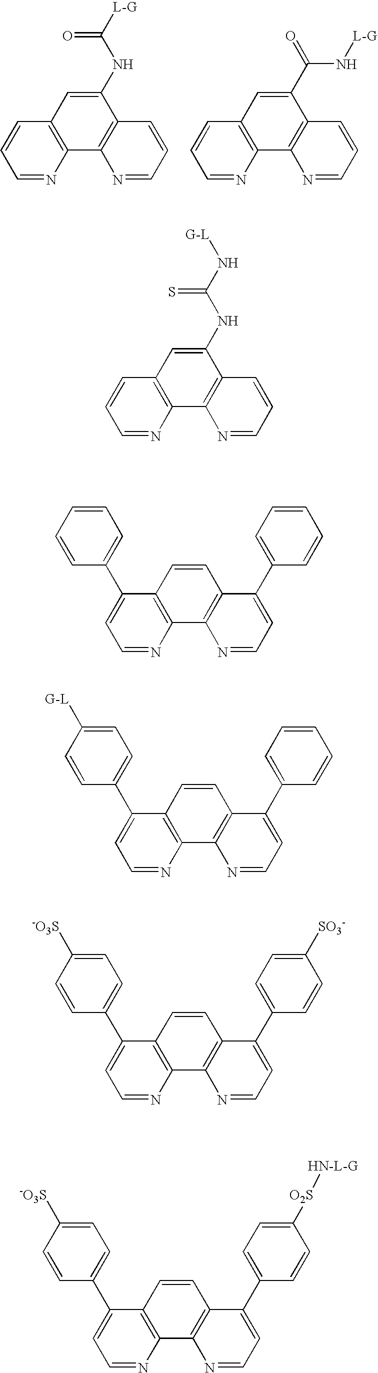

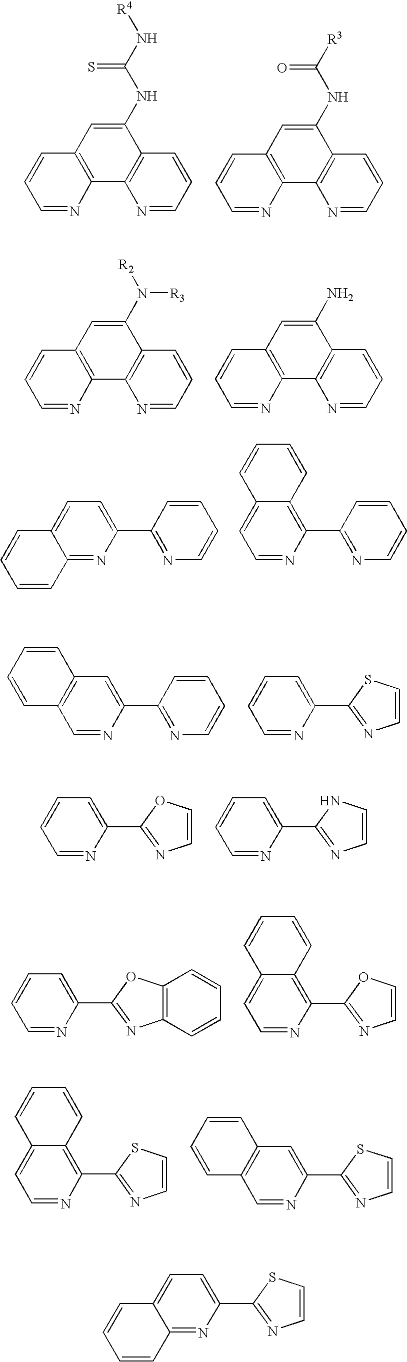

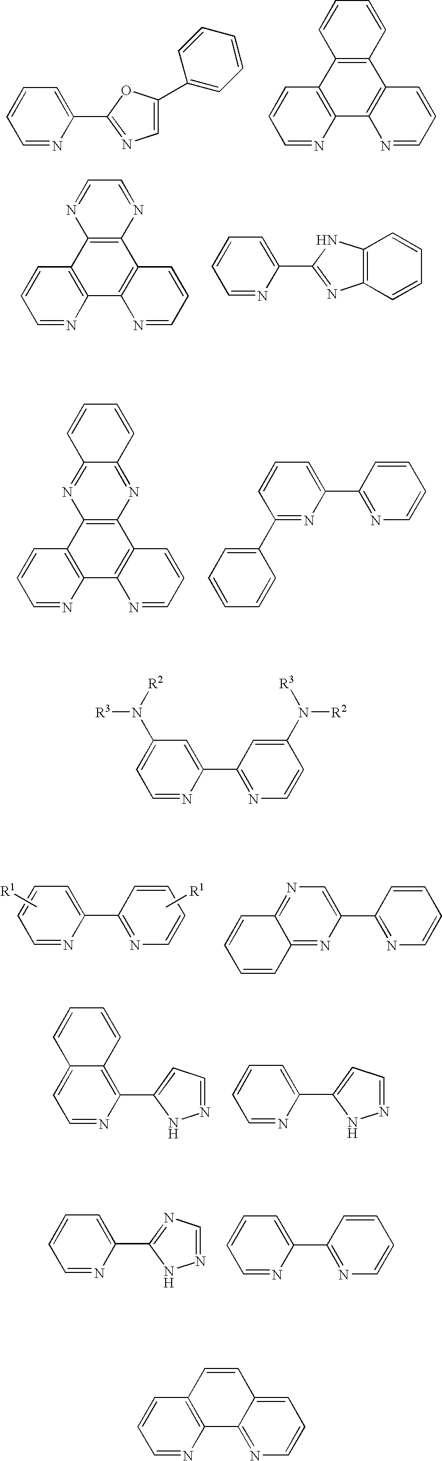

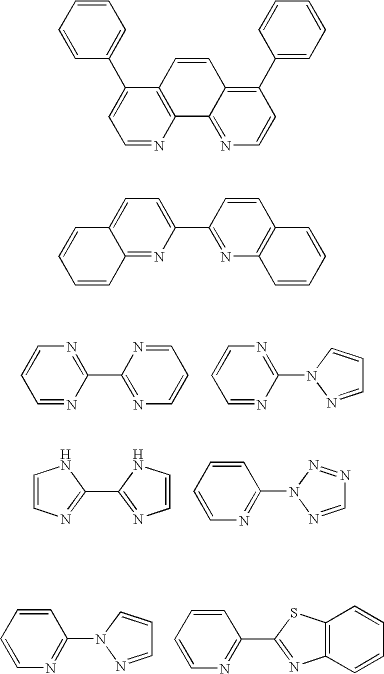

A further example of a generic formula for the nitrogen donor ligand, also showing the possible inclusion of the ligand and reactive group, is as follows:

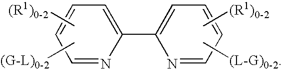

in which each R¹ can be the same or different and R¹ is either C₁₋₆ alkyl, —NR²R³, —N(R²)C(O)R³, —NR²C(O)NR³R⁴, —NR²C(S)NR³R⁴ or phenyl, and R², R³, and R⁴ are each as defined above.

A further example of a generic formula for the nitrogen donor ligand, also showing the possible inclusion of the ligand and reactive group, is as follows:

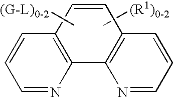

in which each R¹ can be the same or different and R¹ is either C₁₋₆ alkyl, —NR²R³, —N(R²)C(O)R³, —NR²C(O)NR³R⁴, —NR²C(S)NR³R⁴ or phenyl, and R², R³, and R⁴ are each as defined above.

A still further example of a generic formula for the nitrogen donor ligand, also showing the possible inclusion of the ligand and reactive group, is as follows:

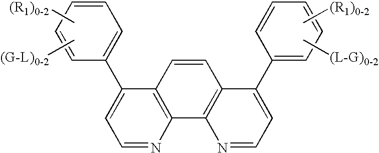

in which each R¹ can be the same or different and R¹ is either C₁₋₆ alkyl, —NR²R³, —N(R²)C(O)R³, —NR²C(O)NR³R⁴, —NR²C(S)NR³R⁴ or phenyl, and R², R³, and R⁴ are each as defined above.

Yet another example of a generic formula for the nitrogen donor ligand is as follows:

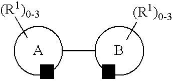

in which ring A contains five to twenty ring atoms, and zero to four of the ring atoms of ring A other than the N atom shown are each either N, O, or S; and ring B contains five to twenty ring atoms, and zero to four of the ring atoms of ring B other than the N atom shown are each either N, O, or S; and R¹ is as defined above. Preferably, rings A and B each contain five to ten ring atoms in addition to the N atoms shown, all such ring atoms except for the N being C atoms.

Preferred heteroaryl ring systems are 2,2′-bipyridine, 1,10-phenanthroline, 4,7-diphenyl-1,10-phenanthroline, 2-(3H pyrrol-2-yl)pyridine, and 2-(pyridin-2-yl)-3H-indole, each substituted with from zero to four halogen atoms. A particularly preferred heteroaryl ring system is 1,10-phenanthroline, substituted with from zero to four halogen atoms.

### Cyclometalated Donor Ligands

Cyclometalated donor ligands useful in the method of the present invention can be any cyclometalated donor ligand that has one nitrogen donor atom and one carbanion available to bind to a metal. In some embodiments, the metal complexes can have 1, 2 or 3 cyclometalated donor ligands. When two or more cyclometalated donor ligands are present in a single metal complex, they can be the same or different. In some embodiments, each cyclometalated donor ligand will have a heteroaryl ring system having from ten to forty ring atoms, where from one to four of the ring atoms are N, O or S, wherein at least one ring atom is N, substituted with from xero to four R¹ groups, where R¹ is either C₁-C₆ alkyl, C₂-C₆ alkenyl, C₂-C₆ alkynyl, C₁-C₆ alkoxy, halogen, C₁-C₆ haloalkyl, —OR², —NR²R³, —CN, —C(O)R², —C(O)OR², —OC(O)R², —C(O)NR²R³, —N(R²)C(O)R³, —OC(O)NR²R³, —N(R²)C(O)OR³, —NR²C(O)NR³R⁴, —NR²C(S)NR³R⁴, —NO₂, ═O, aryl, heteroaryl, cycloalkyl, heterocycloalkyl, carboxylate, alkyl carboxylate, aryl carboxylate, sulfonic acid, alkyl sulfonic acid, aryl sulfonic acid, phosphonic acid, alkyl phosphonic acid, or aryl phosphonic acid. In the above groups, R² is H or C₁-C₁₂ alkyl, R³ is H or C₁-C₁₂ alkyl, and R⁴ is H or C₁-C₁₂ alkyl.

Preferred heteroaryl ring systems for the cyclometalated donor ligand are those having ten to thirty ring atoms and in which each heteroatom is N. Further preferred heteroaryl ring systems are fused ring systems having ten to twenty ring atoms and in which each heteroatom is N and R¹ is halogen.

Examples of generic structures for the cyclometalated donor ligand are as follows, with the nomenclature of the species in which the R groups are H and X is —CH₂—:

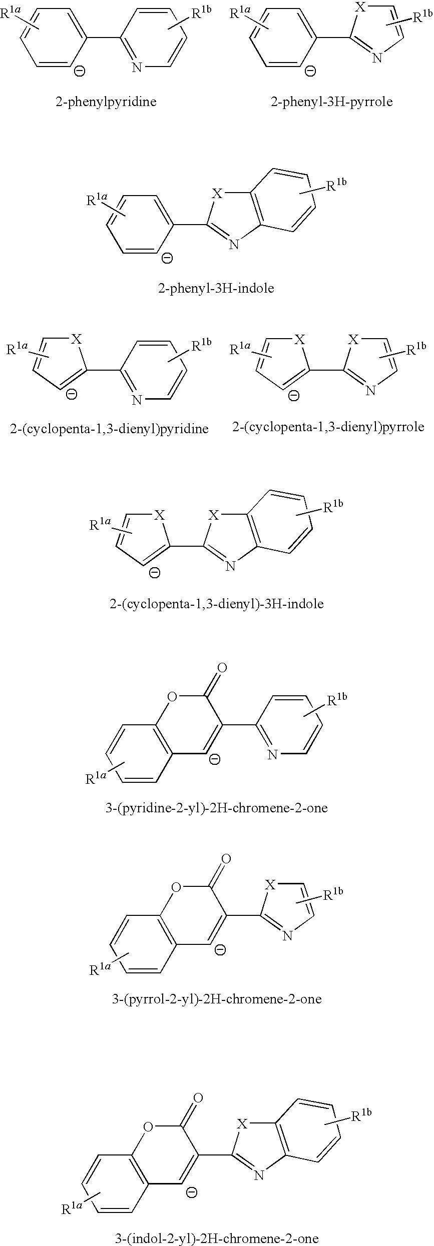

Each of the R groups are as defined above, and X is optionally substituted methylene, O, S, NR², or Se.

Examples of specific cyclometalated ligands are as follows:

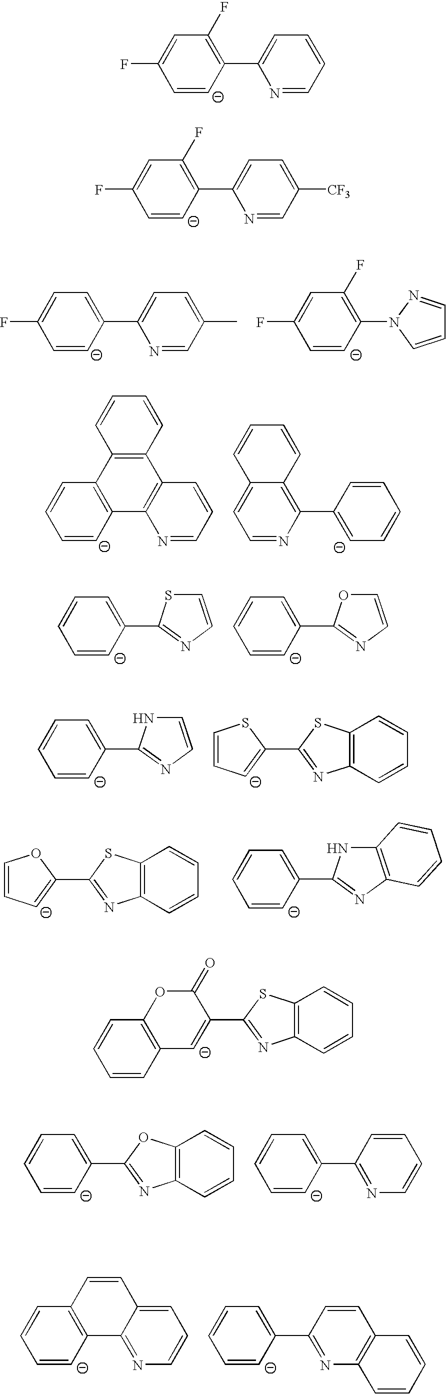

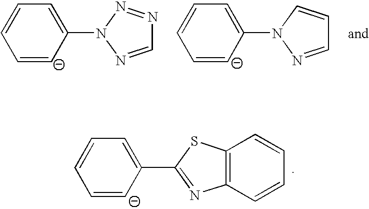

Further examples of generic formulae for the cyclometalated donor ligand are as follows:

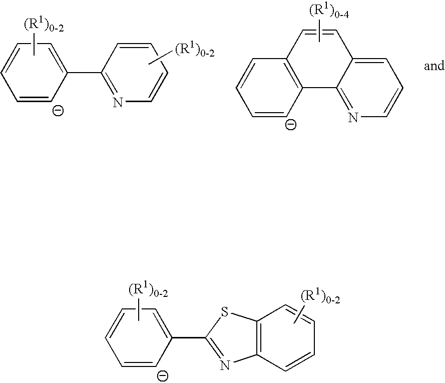

in which R¹ is as defined above.

Yet another example of a generic formula for the cyclometalated donor ligand is as follows:

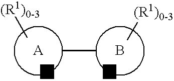

in which ring A contains five to twenty ring atoms, and zero to four of the ring atoms of ring A other than the N atom shown are each either N, O, or S; and ring B contains five to twenty ring atoms, and zero to four of the ring atoms of ring B other than the N atom shown are each either N, O, or S; and R¹ is as defined above. Preferably, rings A and B each contain five to ten ring atoms in addition to the N atoms shown, all such ring atoms except for the N being C atoms.

### Transition Metal Ions

Transition metals that are useful in the present invention include Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg and Ac. The transition metals described above can each adopt several different oxidation states, all of which are useful in the present invention. In some instances, the most stable oxidation state is formed, but other oxidation states are useful in the present invention. Preferred transition metals are Ir, Rh, Os, Pt, Ru, Pd or Re. A particularly preferred transition metal is Ir, notably in the form of Ir(III). A further particularly preferred transition metal is Rh, notably in the form of Rh(III).

### Methods of Using the Complexes

The luminescent complexes of this invention are useful for labeling biomolecules such a proteins and nucleic acids with fluorescent labels, either through covalent bonds or through affinity-type bonds. Labeling can be accomplished by techniques known in the art, including combining the luminescent complex with the biomolecule to be labeled in a solution under conditions favoring reaction between the luminescent complex and the biomolecule. Additional steps to terminate the reaction or to separate unreacted luminescent complex from labeled biomolecule can also be performed. Labeling can occur prior to use of the biomolecules in a test in which a plurality of biomolecules is subjected to a common environment or reactant in order to compare their performance. For instance, proteins can be labeled, then passed through SDS-PAGE electrophoresis, and the resulting protein bands distinguished by their labels. Two or more protein samples, for instance, can be labeled with different complexes and mixed prior to electrophoretic separation. The complexes can also be used in multiplex assays that include immunodetection using antibodies, antibody binders, or affinity-type binding members in general conjugated to the complexes. Oligonucleotides can likewise be conjugated to the complexes for multiplex detection of specific nucleic acid sequences. Still further, the complexes can be used in microscopy or cell-sorting applications.

A particular aspect of this invention involves the use of pairs or sets of the complexes wherein the complexes have structural and chemical features, i.e., physicochemical properties, in common but differ from each other in ways that produce different emission spectra upon excitation. One means by which this is achieved is by the use of complexes that differ only in the substitutions, or in the presence or absence of substitutions, on the donor ligands. Fluorine atoms are examples of substitutions whose presence or absence, and whose number, will produce such a difference. A pair of complexes that are otherwise identical can be used in tandem to detect or identify pairs of proteins, and particularly pairs of related proteins. Once labeled with the complexes, the proteins may be combined and separated in the same medium by various forms of electrophoresis or chromatography.

Thus, two or more samples can be compared for their inclusion of particular proteins of interest, the amounts of those proteins included in each sample, or both, by a sequence that includes first preparing an extract of proteins from each sample, then reacting each extract with a different dye from a set of the dyes described herein to label the proteins in the extracts, where the dyes differ from each other by a difference in one or more of the donor ligands, the differences being such that each dye upon excitation emits luminescent light at a spectrum that is sufficiently different from the spectra of emitted luminescent light from the other dyes to provide each dye with a detectably distinct signal. Once the dye-labeling has been performed on the extracts, the extracts are mixed to form a single combined mixture of dye-labeled proteins, and the proteins of interest are separated from other dye-labeled proteins in the single combined mixture, such as by electrophoretic or other chromatographic means. Common examples are one-dimensional PAGE and two-dimensional PAGE. The separated dye-labeled proteins of interest are then irradiated with excitation energy, and the different dyes are used to distinguish the proteins from one sample from the proteins from the other samples. This can be achieved by a fluorescence scanner that scans at multiple wavelengths, or by a fluorescence microscope, or by the use of multiple filters, or other similar methods known in the art. Scanning and detection can be automated by computer using electronic images. In all of these methods, differences in luminescence intensity between the dye-labeled proteins of the various samples are detected as an indication of differences in presence, amounts, or both, of the proteins of interest among the samples.

Kits for labeling proteins or other molecules contain one or more complexes of this invention, optionally accompanied by other materials. The metal complex may be present as a pure substance or as a stock solution. The kit may contain buffers, chelators, detergents or additional chemical components that facilitate labeling of the target or purification of the labeled target. The kit may additionally contain tubes, columns or chromatographic material.

### Molecules to be Labeled with the Complexes

The reactive groups of the complexes of the present invention are selected to form covalent bonds with a wide range of molecules, many of which are biomolecules. Prominent among biomolecules are proteins and nucleic acids and conservatively modified variants of both amino acid sequences and nucleic acid sequences. Binding to proteins, amino acid sequences, and conservatively modified variants thereof is accomplished by binding to individual amino acids, while binding to nucleic acids and conservatively modified variants thereof is accomplished by binding to individual nucleotides.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid” also refers to poly(amino acids) such as peptides, polypeptides and proteins.

The term “amino acid analogs” refers to compounds that have the same basic chemical structure as a naturally occurring amino acid, i.e., an α-carbon that is bound to a hydrogen, a carboxyl group, an amino group, and an R group. Examples of amino acid analogs include, but are not limited to, homoserine, norleucine, methionine sulfoxide, methionine methyl sulfonium. Such analogs have modified R groups (e.g., norleucine) or modified peptide backbones, but retain the same basic chemical structure as a naturally occurring amino acid.

The term “unnatural amino acids” refers to amino acids that are not encoded by the genetic code and can, but do not necessarily, have the same basic structure as a naturally occurring amino acid. Unnatural amino acids include, but are not limited to, azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, ornithine, pentylglycine, pipecolic acid and thioproline.

The term “amino acid mimetics” refers to chemical compounds that have structures that differ from the general chemical structure of an amino acid, but that function in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

The term “conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid (i.e., hydrophobic, hydrophilic, positively charged, neutral, negatively charged). Examples of hydrophobic amino acids are valine, leucine, isoleucine, methionine, phenylalanine, and tryptophan. Examples of aromatic amino acids are phenylalanine, tyrosine and tryptophan. Examples of aliphatic amino acids are serine and threonine. Examples of basic amino acids are lysine, arginine and histidine. Examples of amino acids with carboxylate side-chains are aspartate and glutamate. Examples of amino acids with carboxamide side chains are asparagines and glutamine. Conservative substitution tables providing functionally similar amino acids are well known in the art. Such conservatively modified variants are in addition to and do not exclude polymorphic variants, interspecies homologs, and alleles of the invention.

The following groups each contain amino acids that are conservative substitutions for one another:

Alanine (A), Glycine (G)
Aspartic acid (D), Glutamic acid (E)
Asparagine (N), Glutamine (Q)
Arginine (R), Lysine (K)
Isoleucine (I), Leucine (L), Methionine (M), Valine (V)
Phenylalanine (F), Tyrosine (Y), Tryptophan (W)
Serine (S), Threonine (T)
Cysteine (C), Methionine (M)

### Further Definitions

The term “alkyl” refers to a straight or branched, saturated, aliphatic radical having the number of carbon atoms indicated. For example, C₁-C₆ alkyl includes, but is not limited to, methyl, ethyl, propyl, butyl, pentyl, hexyl, iso-propyl, iso-butyl, sec-butyl, tert-butyl, etc.

The term “lower” as used in connection with alkyl and other organic radicals or compounds denotes a radical or compound, branched or unbranched, with from 1 to 7, preferably from 1 to 4 carbon atoms, and most preferably, if unbranched, one or two carbon atoms.

The term “alkenyl” refers to either a straight chain or branched hydrocarbon of 2 to 6 carbon atoms, having at least one double bond. Examples of alkenyl groups include, but are not limited to, vinyl, propenyl, isopropenyl, butenyl, isobutenyl, butadienyl, pentenyl or hexadienyl.

The term “alkynyl” refers to either a straight chain or branched hydrocarbon of 2 to 6 carbon atoms, having at least one triple bond. Examples of alkynyl groups include, but are not limited to, acetylenyl, propynyl or butynyl.

The term “alkoxy” refers to alkyl with the inclusion of an oxygen atom, for example, methoxy, ethoxy, etc. “Halo-substituted-alkoxy” is as defined for alkoxy where some or all of the hydrogen atoms are substituted with halogen atoms. For example, halo-substituted-alkoxy includes trifluoromethoxy, etc.

As used herein, the term “cycloalkyl” refers to a saturated or partially unsaturated, monocyclic, fused bicyclic or bridged polycyclic ring assembly containing from 3 to 12 ring atoms, or the number of atoms indicated For example, C₃₋₈ cycloalkyl includes cyclopropyl, cyclobutyl, cyclopentyl, cyclohexyl, and up to cyclooctyl.

The term “heterocycle” refers to a ring system having from 3 ring members to about 20 ring members and from 1 to about 5 heteroatoms such as N, O and S. Additional heteroatoms can also be useful, including, but not limited to, B, Al, Si and P. The heteroatoms can also be oxidized, such as, but not limited to, —S(O)— and —S(O)₂—. For example, heterocycle includes, but is not limited to, tetrahydrofuranyl, tetrahydrothiophenyl, morpholino, pyrrolidinyl, pyrrolinyl, imidazolidinyl, imidazolinyl, pyrazolidinyl, pyrazolinyl, piperazinyl, piperidinyl, indolinyl, quinuclidinyl and 1,4-dioxa-8-aza-spiro[4.5]dec-8-yl.

The term “aryl” refers to a monocyclic or fused bicyclic, tricyclic or greater, aromatic ring assembly containing 6 to 16 ring carbon atoms. For example, aryl may be phenyl, benzyl or naphthyl, preferably phenyl. “Arylene” means a divalent radical derived from an aryl group. Aryl groups can be mono-, di- or tri-substituted by one, two or three radicals selected from alkyl, alkoxy, aryl, hydroxy, halogen, cyano, amino, amino-alkyl, trifluoromethyl, alkylenedioxy and oxy-C₂-C₃-alkylene; all of which are optionally further substituted, for instance as hereinbefore defined; or 1- or 2-naphthyl; or 1- or 2-phenanthrenyl. Alkylenedioxy is a divalent substitute attached to two adjacent carbon atoms of phenyl, for example methylenedioxy or ethylenedioxy. Oxy-C₂-C₃-alkylene is also a divalent substituent attached to two adjacent carbon atoms of phenyl, e.g. oxyethylene or oxypropylene. An example for oxy-C₂-C₃-alkylene-phenyl is 2,3-dihydrobenzofuran-5-yl.

Preferred aryl groups are naphthyl, phenyl and phenyl mono- or disubstituted by alkoxy, phenyl, halogen, alkyl or trifluoromethyl, especially phenyl and phenyl-mono- or disubstituted by alkoxy, halogen or trifluoromethyl, and in particular phenyl.

The term “heteroalkyl” refers to an alkyl group in which one or more of the carbon atoms is replaced by the heteroatom N, O, or S, with appropriate changes in the number of H atoms bonded to the heteroatom.

The term “heteroaryl” refers to a monocyclic or fused bicyclic or tricyclic aromatic ring assembly containing 5 to 16 ring atoms, where from 1 to 4 of the ring atoms are a heteroatom each N, O or S. For example, heteroaryl includes pyridyl, indanyl, imidazolyl, quinoxalinyl, quinolinyl, isoquinolinyl, benzothienyl, benzofuranyl, furanyl, pyrrolyl, thiazolyl, benzothiazolyl, oxazolyl, benzoxazolyl, isoxazolyl, triazolyl, tetrazolyl, pyrazolyl, imidazolyl, thienyl, or any other radicals substituted, especially mono- or di-substituted, by e.g. alkyl, nitro or halogen. Pyridyl represents 2-, 3- or 4-pyridyl, advantageously 2- or 3-pyridyl. Thienyl represents 2- or 3-thienyl. Quinolinyl represents preferably 2-, 3- or 4-quinolinyl. Isoquinolinyl represents preferably 1-, 3- or 4-isoquinolinyl. Benzopyranyl, benzothiopyranyl represents preferably 3-benzopyranyl or 3-benzothiopyranyl, respectively. Thiazolyl represents preferably 2- or 4-thiazolyl, and, most preferred, 4-thiazolyl. Triazolyl is preferably 1-, 2- or 5-(1,2,4-triazolyl). Tetrazolyl is preferably 5-tetrazolyl. Additional heteroaryl compounds include, but are not limited to, chromenone, chromone and coumarin.

Preferably, heteroaryl is pyridyl, indolyl, quinolinyl, pyrrolyl, thiazolyl, isoxazolyl, triazolyl, tetrazolyl, pyrazolyl, imidazolyl, thienyl, furanyl, benzothiazolyl, benzoxazolyl, benzofuranyl, isoquinolinyl, benzothienyl, oxazolyl, indazolyl, 2-oxo-2H-chromenyl, or any of the radicals substituted, especially mono- or di-substituted.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —CN and —NO₂ in a number ranging from zero to (2m′+1), where m′ is the total number of carbon atoms in such radical. R′, R″ and R′″ each independently refer to hydrogen, unsubstituted (C₁-C₈)alkyl and heteroalkyl, unsubstituted aryl, unsubstituted alkyl, alkoxy or thioalkoxy groups, or unsubstituted aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like). Preferably, the substituted alkyl and heteroalkyl groups have from 1 to 4 substituents, more preferably 1, 2 or 3 substituents. Exceptions are those perhaloalkyl groups (e.g., pentafluoroethyl and the like) which are also preferred and contemplated by the present invention.

The term “halogen” refers to fluorine, chlorine, bromine and iodine.

The term “haloalkyl” refers to alkyl as defined above where some or all of the hydrogen atoms are substituted with halogen atoms. Halogen (halo) preferably represents chloro or fluoro, but may also be bromo or iodo. For example, haloalkyl includes trifluoromethyl, fluoromethyl, 1,2,3,4,5-pentafluoro-phenyl, etc. The term “perfluoro” defines a compound or radical which has at least two available hydrogens substituted with fluorine. For example, perfluorophenyl refers to 1,2,3,4,5-pentafluorophenyl, perfluoromethane refers to 1,1,1-trifluoromethyl, and perfluoromethoxy refers to 1,1,1-trifluoromethoxy.

Examples of substituted phenyl groups are 4-chlorophen-1-yl, 3,4-dichlorophen-1-yl, 4-methoxyphen-1-yl, 4-methylphen-1-yl, 4-aminomethylphen-1-yl, 4-methoxyethylamino-methylphen-1-yl, 4-hydroxyethylaminomethylphen-1-yl, 4-hydroxyethyl-(methyl)-amino-methylphen-1-yl, 3-aminomethylphen-1-yl, 4-N-acetylaminomethylphen-1-yl, 4-aminophen-1-yl, 3-aminophen-1-yl, 2-aminophen-1-yl, 4-phenyl-phen-1-yl, 4-(imidazol-1-yl)-phen-1-yl, 4-(imidazol-1-ylmethyl)-phen-1-yl, 4-(morpholin-1-yl)-phen-1-yl, 4-(morpholin-1-ylmethyl)-phen-1-yl, 4-(2-methoxyethylaminomethyl)-phen-1-yl and 4-(pyrrolidin-1-ylmethyl)-phen-1-yl, 4-(thiophenyl)-phen-1-yl, 4-(3-thiophenyl)-phen-1-yl, 4-(4-methylpiperazin-1-yl)-phen-1-yl, and 4-(piperidinyl)-phenyl and 4-(pyridinyl)-phenyl optionally substituted in the heterocyclic ring.

Similarly, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′—NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, unsubstituted (C₁-C₈)alkyl and unsubstituted heteroalkyl, unsubstituted aryl and unsubstituted heteroaryl, unsubstituted (aryl)-(C₁-C₄)alkyl, and unsubstituted (aryl)oxy-(C₁-C₄)alkyl.

Two of the substituents on adjacent atoms of the aryl or heteroaryl ring may optionally be replaced with a substituent of the formula -T-C(O)—(CH₂)_{q}-U-, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ-B-, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 4, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

### EXAMPLES

The following examples are representative of the preparation and use of complexes of this invention. The invention is far broader than these examples, however, and extends to the use of other complexes, spacers, ligands, functional groups and types of molecules that may be labeled using the complexes, as expressed in the claims.

Syntheses of Complexes A and B: Complexes A and B, shown below, are similar in structure, with a similar charge, size and solubility. These similarities afford complexes A and B, and labeled biomolecules to which these complexes are bound, a similar electrophoretic mobility or chromatographic behavior. They each bear an amine-reactive N-hydroxysuccinimide ester group separated from the luminescent group by a 5-carbon linker (derived from adipic acid). In Complex A the donor ligands are unsubstituted whereas in Complex B they are substituted with four fluorine atoms.

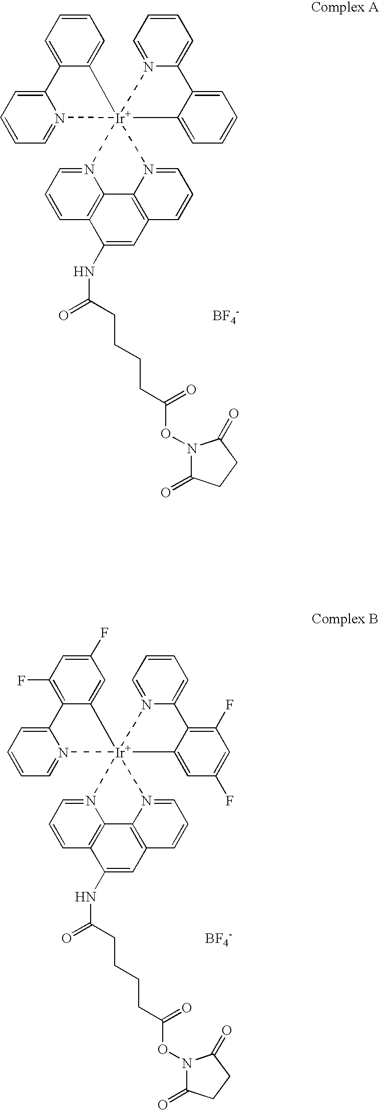

### Synthesis of Complex A

### 1. Synthesis of methyl 5-(1,10-phenanthrolin-5-ylcarbamoyl)pentanoate

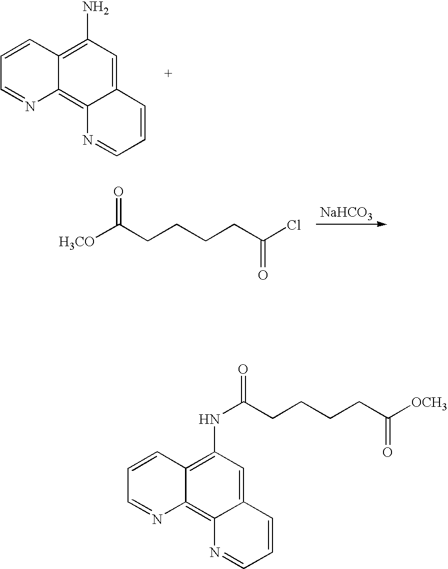

1,10-Phenanthrolin-5-amine (0.488 g) and sodium bicarbonate (0.252 g) were mixed in 30 mL dry acetonitrile and chilled on ice. Methyl adipoyl chloride (584 μL) dissolved in 40 mL dry acetonitrile was added slowly. The mixture was stirred on ice for 4 h. The volume was reduced under vacuum to a few mL and the material was washed with diethyl ether, collected by filtration and dissolved in 200 mL methylene chloride. The solution was filtered and the volume was reduced under vacuum to a few mL. Addition of diethyl ether precipitated the product as an orange gum, which was dried under vacuum, yielding 0.504 g of material (60% yield) which appeared pure by thin-layer chromatography (1:1 methylene chloride:methanol, silica gel).

### 2. Coupling of methyl 5-(1,10-phenanthrolin-5-ylcarbamoyl)pentanoate with 2-phenylpyridine Groups and Iridium Ion and Formation of Succinimidyl Ester

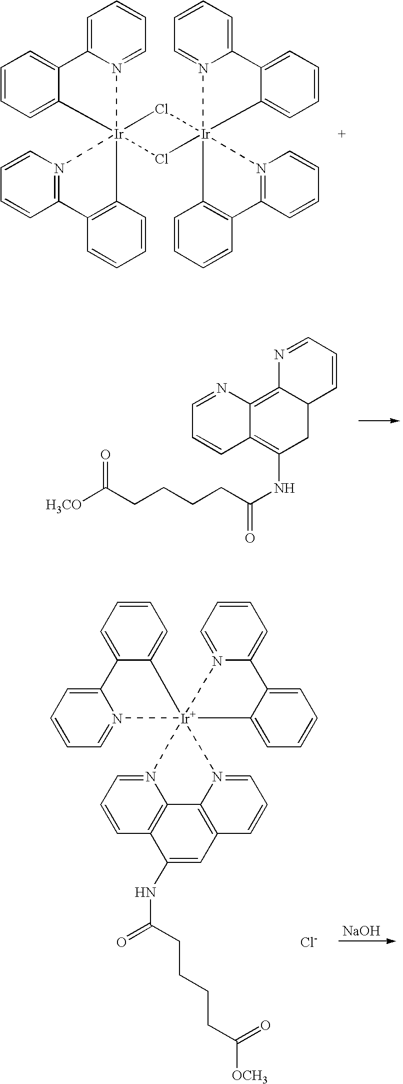

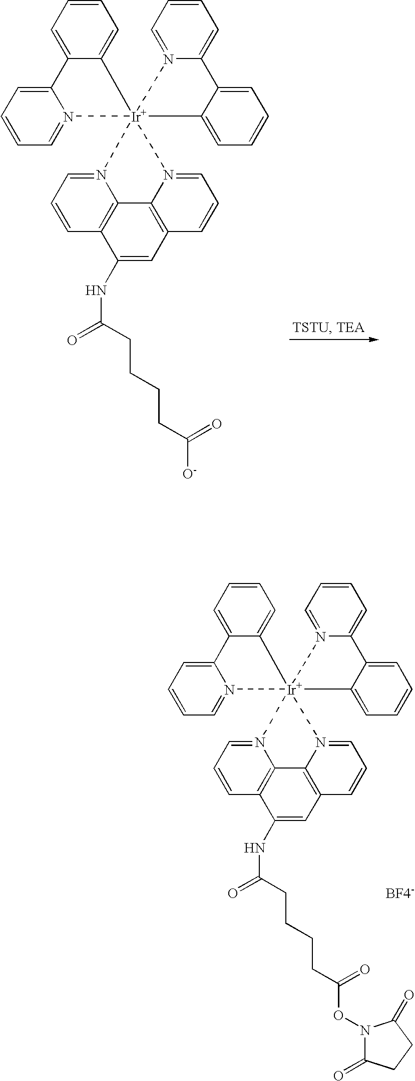

Tetrakis(2-phenylpyridine-C²,N′)(μ-dichloro)diiridium (21.4 mg, Sigma-Aldrich) and methyl 5-(1,10-phenanthrolin-5-ylcarbamoyl)pentanoate (16.8 mg) (prepared as above) were dissolved in 2 mL methylene chloride and stirred under argon for 5 h. The material was dried under a stream of nitrogen, dissolved in 1 ml methanol and mixed with 1 mL 1 M NaOH. The mixture was refluxed for 30 min, acidified with 1 ml formic acid and dried under vacuum. This material was dissolved in chloroform and applied to silica gel chromatography in chloroform plus 1% formic acid and eluted sequentially with chloroform, 1% formic acid with 10%, 20% and 30% ethanol. Fractions containing fluorescent material were combined and dried down, yielding 14.4 mg of material (42% yield) appearing pure by thin-layer chromatography (chloroform:ethanol:formic acid 90:9:1 silica gel). A portion of this material (4.3 mg) was mixed with 4.5 mg N,N,N′,N′-Tetramethyl-O—(N-succinimidyl)uronium tetrafluoroborate (TSTU) and 7.25 μl triethylamine (TEA) in 100 μL dry dimethylformamide. After 30 min of reaction at room temperature, the material was purified by silica gel chromatography using chloroform:ethanol 9:1 as the eluant. The purified material was dried, dissolved in dry dimethylformamide to a concentration of 5 mM and stored at −80° C. Identity of the product was verified by matrix-free laser desorption ionization MS.

### Synthesis of Complex B

### 1. Synthesis of tetrakis(2-[2,4-difluorophenyl]pyridine-c²,n′)(μ-dichloro)diiridium

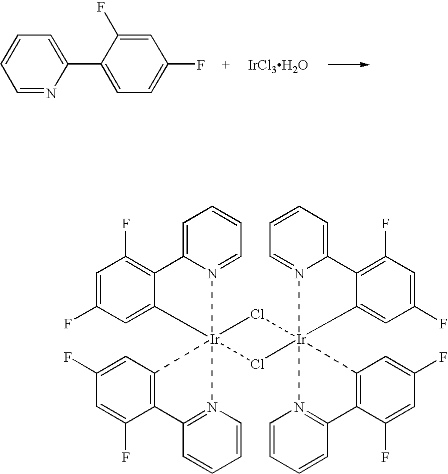

2-(2,4-Difluorophenyl)pyridine (137 μL) and iridium chloride hydrate (75 mg) were refluxed together in 4.5 mL ethoxyethanol and 1.5 mL water for 16 h. The volume was reduced under vacuum to a few mL and precipitated material was collected by filtration, washed with diethyl ether and ethanol and vacuum dried, yielding 130 mg of material (90% yield) that appeared pure by thin-layer chromatography (methanol, silica gel).

### Coupling of methyl 5-(1,10-phenanthrolin-5-ylcarbamoyl)pentanoate with 2,2′-difluorophenylpyridine Groups and Iridium Ion and Formation of Succinimidyl Ester

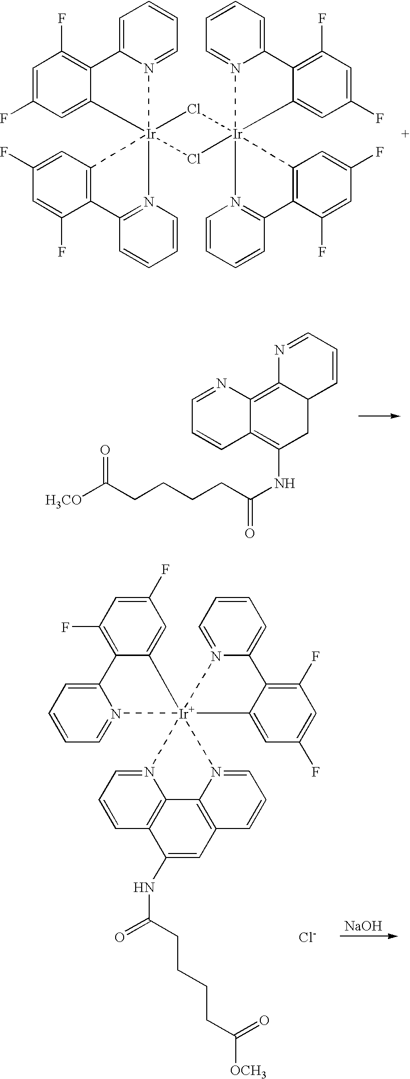

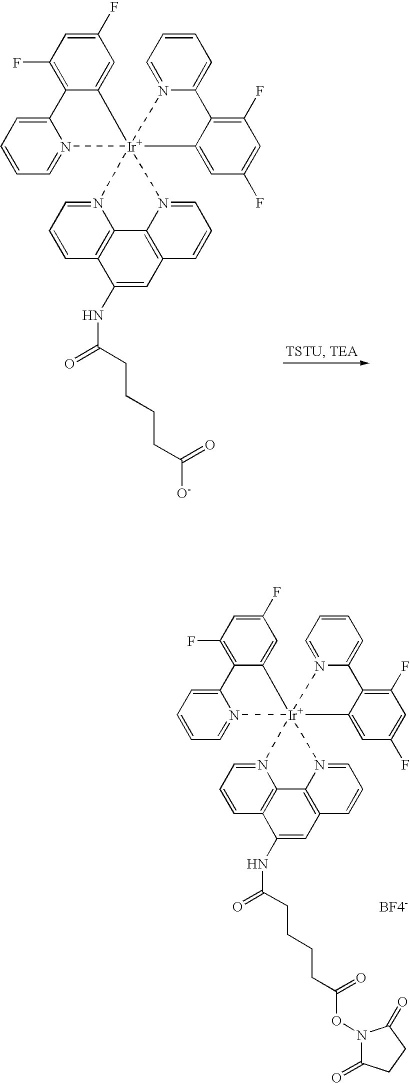

Tetrakis(2-[2,4-difluorophenyl]pyridine-c²,n′)(μ-dichloro)diiridium (24.3, sigma-Aldrich) and methyl 5-(1,10-phenanthrolin-5-ylcarbamoyl)pentanoate (16.8 mg) were dissolved in 2 mL methylene chloride and stirred under argon for 3 h. The material was dried under a stream of nitrogen, dissolved in 1 ml methanol and mixed with 1 mL 1 m NaOH. The mixture was heated at 70° C. for 20 min, acidified with 1 mL formic acid and dried under vacuum. This material was dissolved in chloroform and applied to silica gel chromatography in chloroform plus 1% formic acid and eluted sequentially with chloroform, 1% formic acid with 10%, 20% and 30% ethanol. Fractions containing fluorescent material were combined and dried down, yielding 18.1 mg of material (48% yield) appearing pure by thin-layer chromatography (chloroform:ethanol:formic acid 90:9:1 silica gel). A portion of this material (11.3 mg) was mixed with 10.8 mg n,n,n′,n′-tetramethyl-o-(n-succinimidyl)uronium tetrafluoroborate (TSTU) and 17.4 μL triethylamine (TEA) in 240 μL dry dimethylformamide. After 15 min of reaction at room temperature, the material was purified by silica gel chromatography using chloroform:ethanol 9:1 as the eluant. The purified material was dried, dissolved in dry dimethylformamide to a concentration of 5 mm and stored at −80° C. Identity of the product was verified by matrix-free laser desorption ionization ms.

### Protein Labeling with Complexes A and B and Fluorescence Imaging of Labeled Proteins Following Electrophoretic Separation

A mixture of phosphorylase b (rabbit), serum albumin (bovine), ovalbumin (chicken) and carbonic anhydrase (bovine) at approximately 1 mg/mL each were reacted at room temperature in 25 mM sodium borate buffer pH 8.5 with either Complex A or Complex B at either 100 μM or 10 μM. The mixtures were applied to SDS-PAGE. Following electrophoresis, the gel was photographed with transillumination using UV light centered on a wavelength of approximately 300 nm. Photographs were taken with 300 s exposure either through a 482DF22 nm inferference filter or a 670DF40 nm interference filter.

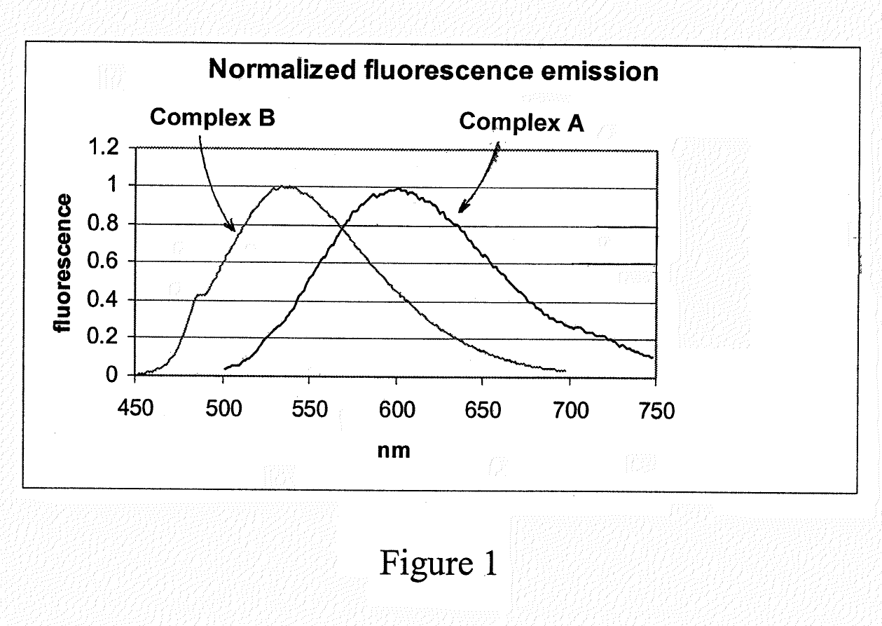

FIG. 1 shows the fluorescence emission spectra of 10 μM of the carboxylic acid forms of Complexes A and B respectively, in 50 mM Tris-Cl (pH 8) with excitation at 240 nm. The spectra have been normalized for equal intensity at the maximum. As can be seen, the emission spectra of the two complexes were different.

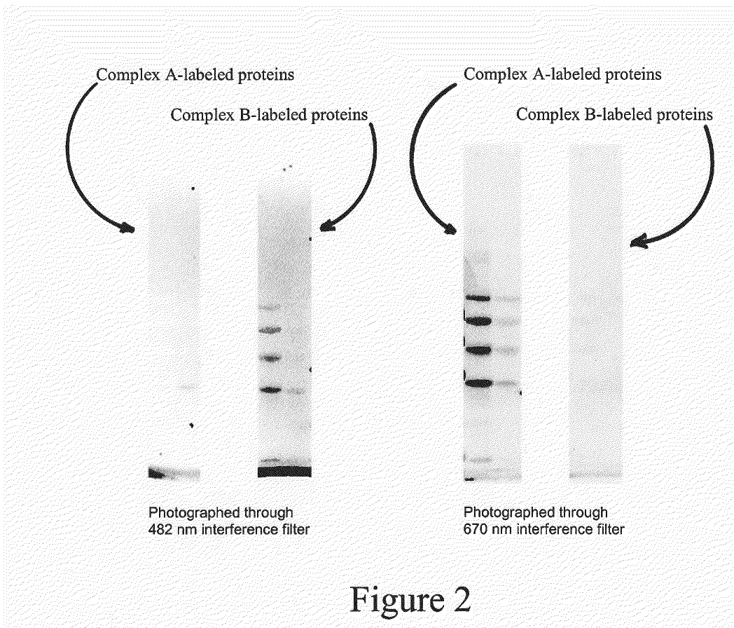

FIG. 2 shows photographs of electrophoretically separated proteins labeled either with Complex A or Complex B. The photographs have been taken through interference filters selected to distinguish between light emitted by either of the two complexes. As can be seen, proteins labeled with either complex can be distinguished from each other.

The foregoing descriptions are offered primarily for purposes of illustration. Further modifications, variations and substitutions that still fall within the spirit and scope of the invention will be readily apparent to those skilled in the art. All such modifications coming within the scope of the appended claims are intended to be included therein.

In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein or any prior art in general and an explicit teaching of this specification is intended to be resolved in favor of the teaching in this specification. This includes any discrepancy between an art-understood definition of a word or phrase and a definition explicitly provided in this specification of the same word or phrase.

What is claimed is:

1. A complex comprising:
a transition metal ion,
a plurality of donor ligands, each said donor ligand fully coordinated to said transition metal ion and each said donor ligand being either a nitrogen donor ligand or a cyclometalated donor ligand, such that at least one of said donor ligands is a cyclometalated donor ligand, and
a reactive group substituted on at least one of said donor ligands through a linker, said reactive group reactive with a functional group to form a covalent bond, and said linker comprising a chain of 4 to 10 atoms.

2. The complex of claim 1 wherein said linker is a member selected from the group consisting of substituted or unsubstituted polymethylene, substituted or unsubstituted arylene, substituted or unsubstituted alkylarylene, substituted or unsubstituted arylenealkyl, and substituted or unsubstituted arylthio.

3. The complex of claim 1 wherein said linker is a member selected from the group consisting of —(CH₂)_{d}(C(O)NH(CH₂)ₑ)_{z}—, —(CH₂)_{d}(C(O)NH(CH₂)₄NH(CH₂)ₑ)_{z}—, —(CH₂)_{d}(C(O)NH(CH₂)ₑNH₂)_{z}—, and —(CH₂)_{d}(C(O)NH(CH₂)ₑNH(CO)_{z}—, where d is zero to 5, e is 1 to 5, and z is zero or 1.

4. The complex of claim 1 wherein said linker is C₂-C₁₀ polymethylene.

5. The complex of claim 1 wherein said linker is C₄-C₁₀ polymethylene.

6. The complex of claim 1 wherein said reactive group is a member selected from the group consisting of a carboxylate, a succinimidyl ester of a carboxylic acid, a haloacetamide, a hydrazine, an isothiocyanate, a maleimido group, an aliphatic amine, a perfluorobenzamido group, an azidoperfluorobenzamido group, a reactive platinum complex, and a psoralen.

7. The complex of claim 1 wherein said reactive group is a member selected from the group consisting of a succinimidyl ester of a carboxylic acid, an iodoacetamide, a maleimido group, and a reactive platinum complex.

8. The complex of claim 1 wherein said reactive group is a succinimidyl-containing group.

9. The complex of claim 1 wherein said reactive group is succinimidooxycarbonyl.

10. The complex of claim 1 wherein said reactive group is succinimidooxycarbonyl, and said linker is C₂-C₁₀ polymethylene.

11. The complex of claim 1 wherein said reactive group is succinimidooxycarbonyl, and said linker is C₂-C₁₀ polymethylene.

12. The complex of claim 1 wherein said reactive group is succinimidooxycarbonyl, and said linker is C₂-C₆ polymethylene.

13. The complex of claim 1 wherein one of said donor ligands is substituted with an isocyanato group, said reactive group is succinimidooxycarbonyl, and said linker is C₂-C₁₀ polymethylene bonded to said isocyanato group.

14. The complex of claim 1 wherein at least one of said donor ligands is a nitrogen donor ligand substituted with an isocyanato group, said reactive group is succinimidooxycarbonyl, and said linker is C₂-C₁₀ polymethylene bonded to said isocyanato group.

15. The complex of claim 1 wherein at least one of said donor ligands is a nitrogen donor ligand substituted with an isocyanato group, said reactive group is succinimidooxycarbonyl, and said linker is C₄-C₁₀ polymethylene bonded to said isocyanato group.

16. The complex of claim 1 wherein at least one of said donor ligands is a nitrogen donor ligand substituted with an isocyanato group, said reactive group is succinimidooxycarbonyl, and said linker is C₂-C₆ polymethylene bonded to said isocyanato group.

17. The complex of claim 1 wherein at least one of said donor ligands is a nitrogen donor ligand substituted with an isocyanato group, said reactive group is succinimidooxycarbonyl, and said linker is C₄ polymethylene bonded to said isocyanato group.

18. The complex of claim 1 wherein:
at least one of said donor ligands is a nitrogen donor ligand containing a heteroaryl ring system having from ten to forty ring atoms, wherein from two to eight of said ring atoms are heteroatoms selected from the group consisting of N, O, S, and combinations thereof, and at least two of said heteroatoms are N, and
said nitrogen donor ligand is substituted with from zero to four R¹ groups, where
R¹ is a member selected from the group consisting of C₁₋₆ alkyl, C₂₋₆ alkenyl, C₂₋₆ alkynyl, C₁₋₆ alkoxy, halogen, C₁₋₆ haloalkyl, —OR², —NR²R³, —CN, —C(O)R², —C(O)OR², —OC(O)R², —C(O)NR²R³, —N(R²)C(O)R³, —OC(O)NR²R³, —N(R²)C(O)OR³, —NR²C(O)NR³R⁴, —NR²C(S)NR³R⁴, —NO₂, ═O, aryl, heteroaryl, cycloalkyl, heterocycloalkyl, carboxylate, alkyl carboxylate, aryl carboxylate, sulfonic acid, alkyl sulfonic acid, aryl sulfonic acid, phosphonic acid, alkyl phosphonic acid and aryl phosphonic acid, where
R² is H or C₁₋₁₂ alkyl,
R³ is H or C₁₋₁₂ alkyl, and
R⁴ is H or C₁₋₁₂ alkyl.

19. The complex of claim 18 wherein said heteroaryl ring system has from ten to thirty ring atoms and each said heteroatom is N.

20. The complex of claim 18 wherein said heteroaryl ring system is a fused ring system having from ten to twenty ring atoms, each said heteroatom is N, and R¹ is halogen.

21. The complex of claim 18 wherein said heteroaryl ring system is a member selected form the group consisting of 2,2′-bipyridine, 1,10-phenanthroline, 4,7-diphenyl-1,10-phenanthroline, 2-(3H pyrrol-2-yl)pyridine, and 2-(pyridin-2-yl)-3H-indole, each substituted with from zero to four halogen atoms.

22. The complex of claim 18 wherein said heteroaryl ring system is 1,10-phenanthroline substituted with from zero to four halogen atoms.

23. The complex of claim 18 said reactive group is substituted on said nitrogen donor ligand through said linker.

24. The complex of claim 18 wherein at least one of said donor ligands is a nitrogen donor ligand having the formula 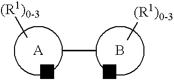
wherein:
ring A contains five to twenty ring atoms, of which zero to four of said ring atoms other than the N atom shown are each either N, O, or S,
ring B contains five to twenty ring atoms, of which zero to four of said ring atoms other than the N atom shown are each either N, O, or S, and
R¹ is a member selected from the group consisting of C₁₋₆ alkyl, C₂₋₆ alkenyl, C₂₋₆ alkynyl, C₁₋₆ alkoxy, halogen, C₁₋₆ haloalkyl, —OR², —NR²R³, —CN, —C(O)R², —C(O)OR², —OC(O)R², —C(O)NR²R³, —N(R²)C(O)R³, —OC(O)NR²R³, —N(R²)C(O)OR³, —NR²C(O)NR³R⁴, —NR²C(S)NR³R⁴, —NO₂, ═O, aryl, heteroaryl, cycloalkyl, heterocycloalkyl, carboxylate, alkyl carboxylate, aryl carboxylate, sulfonic acid, alkyl sulfonic acid, aryl sulfonic acid, phosphonic acid, alkyl phosphonic acid and aryl phosphonic acid, where
R² is H or C₁₋₁₂ alkyl,
R³ is H or C₁₋₁₂ alkyl, and
R⁴ is H or C₁₋₁₂ alkyl.

25. The complex of claim 1 wherein at least one of said donor ligands is a nitrogen donor ligand having the formula 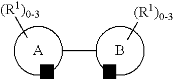
wherein:
ring A contains five to ten ring atoms in addition to the N atom shown, said additional ring atoms being C atoms,
ring B contains five to ten ring atoms in addition to the N atom shown, said additional ring atoms being C atoms, and
R¹ is halogen.

26. The complex of claim 1 wherein:
said cyclometalated donor ligand contains a heteroaryl ring system having from ten to forty ring atoms, wherein from one to four of said ring atoms are heteroatoms selected from the group consisting of N, O, S, and combinations thereof, and at least one of said hetero atoms is N, and
said cyclometalated donor ligand is substituted with from zero to four R¹ groups, where
R¹ is a member selected from the group consisting of C₁₋₆ alkyl, C₂₋₆ alkenyl, C₂₋₆ alkynyl, C₁₋₆ alkoxy, halogen, C₁₋₆ haloalkyl, —OR², —NR²R³, —CN, —C(O)R², —C(O)OR², —OC(O)R², —C(O)NR²R³, —N(R²)C(O)R³, —OC(O)NR²R³, —N(R²)C(O)OR³, —NR²C(O)NR³R⁴, —NR²C(S)NR³R⁴, —NO₂, ═O, aryl, heteroaryl, cycloalkyl, heterocycloalkyl, carboxylate, alkyl carboxylate, aryl carboxylate, sulfonic acid, alkyl sulfonic acid, aryl sulfonic acid, phosphonic acid, alkyl phosphonic acid and aryl phosphonic acid, where
R² is H or C₁₋₁₂ alkyl,
R³ is H or C₁₋₁₂ alkyl, and
R⁴ is H or C₁₋₁₂ alkyl.

27. The complex of claim 26 wherein said heteroaryl ring system has from ten to thirty ring atoms and each said heteroatom is N.

28. The complex of claim 26 wherein said heteroaryl ring system has from ten to twenty ring atoms, each said heteroatom is N, and R¹ is halogen.

29. The complex of claim 26 wherein said heteroaryl ring system is a member selected form the group consisting of 2-phenylpyridine, 2-phenyl-3H-pyrrole, 2-phenyl-3H-indole, 2-(cyclopenta-1,3-dienyl)pyridine, 2-(cyclopenta-1,3-dienyl)pyrrole, 2-(cyclopenta-1,3-dienyl)-3H-indole, 3-(pyridin-2-yl)-2H-chromene-2-one, 3-(3H-pyrrol-2-yl)-2H-chromene-2-one, and 3-(3H-indol-2-yl)-2H-chromene-2-one, each substituted with from zero to four halogen atoms.

30. The complex of claim 26 wherein said heteroaryl ring system is a 2-phenylpyridine ring system, substituted with from zero to four F atoms.

31. The complex of claim 1 wherein said cyclometalated donor ligand is a ligand having the formula 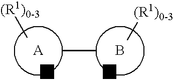
wherein:
ring A contains five to twenty ring atoms, of which zero to four of said ring atoms other than the N atom shown are each either N, O, or S,
ring B contains five to twenty ring atoms, of which zero to four of said ring atoms other than the N atom shown are each either N, O, or S, and
R¹ is a member selected from the group consisting of C₁₋₆ alkyl, C₂₋₆ alkenyl, C₂₋₆ alkynyl, C₁₋₆ alkoxy, halogen, C₁₋₆ haloalkyl, —OR², —NR²R³, —CN, —C(O)R², —C(O)OR², —OC(O)R², —C(O)NR²R³, —N(R²)C(O)R³, —OC(O)NR²R³, —N(R²)C(O)OR³, —NR²C(O)NR³R⁴, —NR²C(S)NR³R⁴, —NO₂, ═O, aryl, heteroaryl, cycloalkyl, heterocycloalkyl, carboxylate, alkyl carboxylate, aryl carboxylate, sulfonic acid, alkyl sulfonic acid, aryl sulfonic acid, phosphonic acid, alkyl phosphonic acid and aryl phosphonic acid, where
R² is H or C₁₋₁₂ alkyl,
R³ is H or C₁₋₁₂ alkyl, and
R⁴ is H or C₁₋₁₂ alkyl.

32. The complex of claim 1 wherein said cyclometalated donor ligand is a ligand having the formula 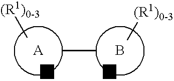
wherein:
ring A contains five to ten ring atoms in addition to the N atom shown, said additional ring atoms being C atoms,
ring B contains five to ten ring atoms in addition to the N atom shown, said additional ring atoms being C atoms, and
R¹ is halogen.

33. The complex of claim 1 wherein:
at least one of said donor ligands is a nitrogen donor ligand containing a first heteroaryl ring system having from ten to forty ring atoms and from two to eight of said ring atoms on said first heteroaryl ring system are heteroatoms, and
R¹ is a member selected from the group consisting of C₁₋₆ alkyl, C₂₋₆ alkenyl, C₂₋₆ alkynyl, C₁₋₆ alkoxy, halogen, C₁₋₆ haloalkyl, —OR², —NR²R³, —CN, —C(O)R², —C(O)OR², —OC(O)R², —C(O)NR²R³, —N(R²)C(O)R³, —OC(O)NR²R³, —N(R²)C(O)OR³, —NR²C(O)NR³R⁴, —NR²C(S)NR³R⁴, —NO₂, ═O, aryl, heteroaryl, cycloalkyl, heterocycloalkyl, carboxylate, alkyl carboxylate, aryl carboxylate, sulfonic acid, alkyl sulfonic acid, aryl sulfonic acid, phosphonic acid, alkyl phosphonic acid and aryl phosphonic acid, where
R² is H or C₁₋₁₂ alkyl,
R³ is H or C₁₋₁₂ alkyl, and
R⁴ is H or C₁₋₁₂ alkyl.

34. The complex of claim 33 wherein said first heteroaryl ring system has from ten to thirty ring atoms, and said second heteroaryl ring system has from ten to thirty ring atoms and each said heteroatom is N.

35. The complex of claim 33 wherein said first heteroaryl ring system is a fused ring system having from ten to twenty ring atoms, said heteroaryl ring system has from ten to twenty ring atoms, each said heteroatom is N, and R¹ is halogen.

36. The complex of claim 33 wherein said first heteroaryl ring system is a member selected form the group consisting of 2,2′-bipyridine, 1,10-phenanthroline, 4,7-diphenyl-1,10-phenanthroline, 2-(3H pyrrol-2-yl)pyridine, and 2-(pyridin-2-yl)-3H-indole, each substituted with from zero to four halogen atoms, and said second heteroaryl ring system is a member selected form the group consisting of 2-phenylpyridine, 2-phenyl-3H-pyrrole, 2-phenyl-3H-indole, 2-(cyclopenta-1,3-dienyl)pyridine, 2-(cyclopenta-1,3-dienyl)pyrrole, 2-(cyclopenta-1,3-dienyl)-3H-indole, 3-(pyridin-2-yl)-2H-chromene-2-one, 3-(3H-pyrrol-2-yl)-2H-chromene-2-one, and 3-(3H-indol-2-yl)-2H-chromene-2-one, each substituted with from zero to four halogen atoms.

37. The complex of claim 33 wherein said first heteroaryl ring system is 1,10-phenanthroline, and said heteroaryl ring system is 2-phenylpyridine substituted with from zero to four F atoms.

38. The complex of claim 1 wherein said plurality of donor ligands consists of one said nitrogen donor ligand and two said cyclometalated donor ligands.

39. The complex of claim 1 wherein said transition metal ion is a member selected from the group consisting of Ir, Rh, Os, Pt, Ru, Pd, and Re.

40. The complex of claim 1 wherein said transition metal ion is Ir(III).

41. The complex of claim 1 wherein:
said plurality of donor ligands consists of one said nitrogen donor ligand and two said cyclometalated donor ligands,
said nitrogen donor ligand is 2-isocyanato-1,10-phenanthroline,
said cyclometalated donor ligands are 2-phenylpyridine substituted with from zero to four F atoms,
said reactive group is succinimidooxycarbonyl,
said linker is C₂-C₁₀ polymethylene, and
said transition metal ion is a member selected from the group consisting of Ir, Rh, Os, Pt, Ru, Pd, and Re.

42. The complex of claim 1 wherein:
said plurality of donor ligands consists of one said nitrogen donor ligand and two said cyclometalated donor ligands,
said nitrogen donor ligand is 2-isocyanato-1,10-phenanthroline,
said cyclometalated donor ligands are 2-phenylpyridine,
said reactive group is succinimidooxycarbonyl,
said linker is —(CH₂)₄—, and
said transition metal ion is Ir (III).

43. The complex of claim 1 wherein:
said plurality of donor ligands consists of one said nitrogen donor ligand and two said cyclometalated donor ligands,
said nitrogen donor ligand is 2-isocyanato-1,10-phenanthroline,
said cyclometalated donor ligands are 2-phenylpyridine, one such 2-phenylpyridine substituted with four F atoms,
said reactive group is succinimidooxycarbonyl,
said linker is —(CH₂)₄—, and
said transition metal ion is Ir (III).

44. A kit for labeling proteins, said kit comprising:
a plurality of complexes, each complex comprising a transition metal ion; a plurality of donor ligands, each said donor ligand fully coordinated to said transition metal ion and each said donor ligand being either a nitrogen donor ligand or a cyclometalated donor ligand, such that at least one of said donor ligands is a cyclometalated donor ligand; and a reactive group substituted on at least one of said donor ligands through a linker, said reactive group reactive with a functional group to form a covalent bond, and said linker comprising a chain of 4 to 10 atoms;
said complexes differentiable from each other by luminescent emissions emitted by said cyclometalated transition metal complexes upon excitation.

45. A method for individually detecting a plurality of proteins in a single liquid sample, said method comprising:
(a) incubating said sample with a plurality of cyclometalated transition metal complexes that are equal in number to said proteins and that are selected such that said cyclometalated transition metal complexes are differentiable from each other by luminescent emissions emitted by said cyclometalated transition metal complexes upon excitation, each said cyclometalated transition metal complex further having bonded thereto an immunological binding member having selective binding affinity toward one of said proteins, to cause each protein to bind to a different cyclometalated transition metal complex by affinity binding through said immunological binding member; and
(b) with said proteins so bound, detecting cyclometalated transition metal complexes bound to said proteins while differentiating said cyclometalated transition metal complexes so detected by luminescent emissions, thereby individually detecting said analytes;
each said cyclometalated transition metal complex comprising:
a transition metal ion,
a plurality of donor ligands, each said donor ligand fully coordinated to said transition metal ion and each said donor ligand being either a nitrogen donor ligand or a cyclometalated donor ligand, such that at least one of said donor ligands is a cyclometalated donor ligand, and
a reactive group substituted on at least one of said donor ligands through a linker, said reactive group reactive with a functional group to form a covalent bond, and said linker comprising a chain of 4 to 10 atoms.

46. A method for comparing a plurality of samples for their contents of selected proteins defined as proteins of interest, said method comprising:
(a) preparing an extract of proteins from each of said samples;
(b) reacting each said extract with a different dye from a set of matched luminescent dyes to form dye-labeled proteins, wherein each said luminescent dye comprises:
a transition metal ion,
a plurality of donor ligands, each said donor ligand fully coordinated to said transition metal ion and each said donor ligand being either a nitrogen donor ligand or a cyclometalated donor ligand, such that at least one of said donor ligands is a cyclometalated donor ligand, each dye differing from the other dyes in said set by a difference in a donor ligand, said difference causing each dye of said set upon excitation to emit luminescent light at a spectrum that is sufficiently different from the spectra of emitted luminescent light from the remaining dyes of said set to provide each dye with a detectably distinct signal, and
a reactive group substituted on at least one of said donor ligands through a linker, said reactive group reactive with one of said proteins to bind said protein to said donor ligand, optionally through an affinity binding member, said linker comprising a chain of 4 to 10 atoms;
(c) mixing said extracts resulting from step (b) to form a single combined mixture of dye-labeled proteins;
(d) separating dye-labeled proteins of interest from other dye-labeled proteins in said single combined mixture; and
(e) irradiating said separated dye-labeled proteins of interest with excitation energy and detecting differences in luminescence intensity between the dye-labeled proteins of interest labeled with one dye and the dye-labeled proteins of interest labeled with other dyes as an indication of differences in amounts of said proteins of interest among said samples.

47. A method of detecting an analyte in a sample, said method comprising
(a) contacting said sample with a luminescent complex comprising:
a transition metal ion,
a plurality of donor ligands, each said donor ligand fully coordinated to said transition metal ion and each said donor ligand being either a nitrogen donor ligand or a cyclometalated donor ligand, such that at least one of said donor ligands is a cyclometalated donor ligand, and
a binding member selected from the group consisting of an antibody, an oligonucleotide, and an affinity-type binding reagent other than antibodies and oligonucleotides, and covalently attached to one of said donor ligands through a linker comprising a chain of 4 to 10 atoms, said binding member being interactive with said analyte upon contact to bind thereto by either a covalent bond or an affinity-type interaction; and
(b) isolating components of said sample to which said luminescent complex is bound in step (b) from unbound molecules of said luminescent complex, and
(c) irradiating said sample with excitation energy and detecting luminescence emitted therefrom as an indication of the presence of said analyte in said sample.
